(19) United States
(12) Patent Application Publication
(10) Pub. No.: US 2015/0050735 A1
(43) Pub. Date: Feb. 19, 2015

(54) WELL PLATES

(71) Applicant: 3-D MATRIX, LTD., Tokyo (JP)

(72) Inventors: Solomon Iyasere, Cambridge, MA (US); Meagan Lazor, Woburn, MA (US); Eun Seok Gil, Lexington, MA (US); Manav Mehta, Cambridge, MA (US)

(21) Appl. No.: 14/459,816

(22) Filed: Aug. 14, 2014

Related U.S. Application Data
(60) Provisional application No. 61/865,918, filed on Aug. 14, 2013.

(51) Int. Cl. C12M 1/32; C12M 1/12; C12N 5/00

(52) U.S. Cl. CPC C12M 23/12; C12N 5/0062; C12M 25/06; C12N 2513/00
USPC 435/395; 435/305.2; 435/325

(57) ABSTRACT

Well plates for use in various laboratory experiments may include a plurality of wells each having an interior wall that includes a ledge positioned at a fixed depth within the well.

### CROSS-REFERENCE TO RELATED APPLICATION

This application claims the benefit of priority to U.S. Provisional Patent Application Ser. No. 61/865,918, filed on Aug. 14, 2013, the entire disclosure of which is hereby incorporated herein by reference in its entirety for all purposes.

### FIELD OF THE TECHNOLOGY

One or more aspects relate generally to well plates for use in laboratory experiments.

### BACKGROUND

Well plates are a conventional tool in various forms of analytical research. Cell culture experiments, for example, generally involve growing cells on a substrate in a well of a well plate under controlled conditions. Media is changed regularly to provide fresh nutrients. Three-dimensional (3D) cell culture is an emerging platform technology that allows for increased cell proliferation, differentiation, and function.

### SUMMARY

One or more aspects relate generally to well plates for use in various laboratory experiments.

In accordance with one or more aspects, a well plate may include a plurality of wells, each well having an interior wall that includes a ledge positioned at a fixed depth within the well to facilitate media changes during a cell culture experiment.

In some aspects, the fixed depth may be related to a predetermined volume of media used during the cell culture experiment. In some non-limiting aspects, the fixed depth is about 0.1 mm to about 10 mm from a bottom of the well. The ledge may be constructed and arranged to deter substrate disruption during media changes. The ledge may protrude into the well along at least a portion of a circumference of the interior wall. In some aspects, the ledge may protrude a uniform distance into the well along at least a portion of the circumference of the interior wall. In some non-limiting aspects, the ledge may protrude about 0.5 mm to about 5 mm into the well along at least a portion of the circumference of the interior wall. In at least some aspects, the ledge may protrude into the well along an entire circumference of the interior wall to define a first well zone above the ledge and a second well zone below the ledge. The second well zone may have a smaller diameter than the first well zone. The second well zone may have a substantially flat bottom and may be configured to receive a substrate during the cell culture experiment. In some aspects, the ledge may define a substantially cylindrical step in the well between the first and second well zones. In at least some aspects, the ledge is configured to provide a platform for a pipette tip in a location that is nonadjacent to the substrate during the cell culture experiment. In some non-limiting aspects, the well plate may include 6, 12, 24, 48, 96, 192, 384, or 1536 wells.

In accordance with one or more aspects, a cell culture kit may include a well plate having a plurality of wells, each well having an interior wall that includes a ledge positioned at a fixed depth within the well to facilitate media changes during a cell culture experiment. The cell culture kit may further include a substrate and instructions for conducting a media change in a well of the well plate while deterring disruption of the substrate during a cell culture experiment. In some aspects, the kit may further include a source of media. In some non-limiting aspects, the substrate may be a hydrogel that includes a repeating amino acid sequence of Arginine-Alanine-Aspartic Acid-Alanine.

In accordance with one or more aspects, a method of changing media in a well of a well plate during a cell culture experiment may involve introducing a pipette tip to the well, tracing an interior wall of the well with the pipette tip from a top of the well to a ledge positioned at a fixed depth within the well, resting the pipette tip on the ledge, aspirating a predetermined volume of spent media from the well with the pipette tip based on the fixed depth of the ledge, and introducing a predetermined volume of fresh media to the well without disrupting a substrate located nonadjacent to the ledge within the well.

In accordance with one or more aspects, a method of facilitating cell culture may involve providing a well plate including a plurality of wells, each well having an interior wall that includes a ledge positioned at a fixed depth within the well to facilitate media changes during a cell culture experiment. The method may further involve providing instructions for conducting a media change in a well of the well plate during a cell culture experiment. In some aspects, the method may still further involve providing a substrate for use during the cell culture experiment. In accordance with one or more aspects, a well plate accessory may comprise an insert for a well of a well plate. The insert may be constructed and arranged to provide a ledge at a fixed depth within the well to facilitate media changes during a cell culture experiment.

Still other aspects, embodiments, and advantages of these exemplary aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. The accompanying drawings are included to provide illustration and a further understanding of the various aspects and embodiments, and are incorporated in and constitute a part of this specification. The drawings, together with the remainder of the specification, serve to explain principles and operations of the described and claimed aspects and embodiments.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the disclosed embodiments, and are not intended as a definition of the limits of such embodiments. For purposes of clarity, not every component may be labeled in every drawing. In the following description, various embodiments are described with reference to the following drawings, in which:

FIG. 1 presents an angled view of a well plate in accordance with one or more embodiments;

FIG. 2 presents a front view of a well plate in accordance with one or more embodiments;

FIG. 3 presents an angled view of a single well of a well plate in accordance with one or more embodiments;

FIG. 4 presents a top view of a well plate in accordance with one or more embodiments;

FIGS. 5A-5B present schematic views of potential ledge contours for a well of a well plate in accordance with one or more embodiments;

FIG. 6 presents an angled view of an insert in accordance with one or more embodiments;

FIG. 7 presents a bottom angled view of an insert in accordance with one or more embodiments;

FIG. 8 presents a front view of an insert in accordance with one or more embodiments;

FIG. 9 presents a right side view of an insert in accordance with one or more embodiments;

FIG. 10 presents a top view of an insert in accordance with one or more embodiments;

FIG. 11 presents an insert placed in a well of a well plate in accordance with one or more embodiments;

FIG. 12 presents an angled view of a well plate filled with inserts in accordance with one or more embodiments;

FIG. 13 presents a bottom angled view of a well plate filled with inserts in accordance with one or more embodiments;

FIG. 14 presents a side view of a well plate filled with inserts in accordance with one or more embodiments; and

FIG. 15 presents a top view of a well plate filled with inserts in accordance with one or more embodiments.

### DETAILED DESCRIPTION

In accordance with one or more embodiments, a well plate may facilitate laboratory experiments, such as cell culture experiments, to increase user confidence. One or more embodiments may be particularly beneficial for 3D cell culture, however, broader applications exist for general cell culture as well as other forms of analytical research. Embodiments may address difficulties associated with the fragility of various substrates, such as hydrogels, used in 3D cell culture. Substrate disruption is common with regular media changes that are required in order to maintain cell health and may be prevented in at least some embodiments. 3D cell culture typically involves more frequent media changes than conventional two-dimensional (2D) cell culture. Embodiments may also facilitate accurate volumetric measurement of media for consistent signaling factors. Embodiments may largely remove the guess work, stress and human error associated with routine media changes by significantly reducing the chance of substrate disruption as well as by allowing for a predetermined amount of media to remain on top of cells during the process of media changes. Thus, all wells of a well plate may generally have the same volume of remaining media during a media change which will provide consistency of signaling factors across all wells of the well plate. The well plates may also interface with automated robotic processes used in experimentation.

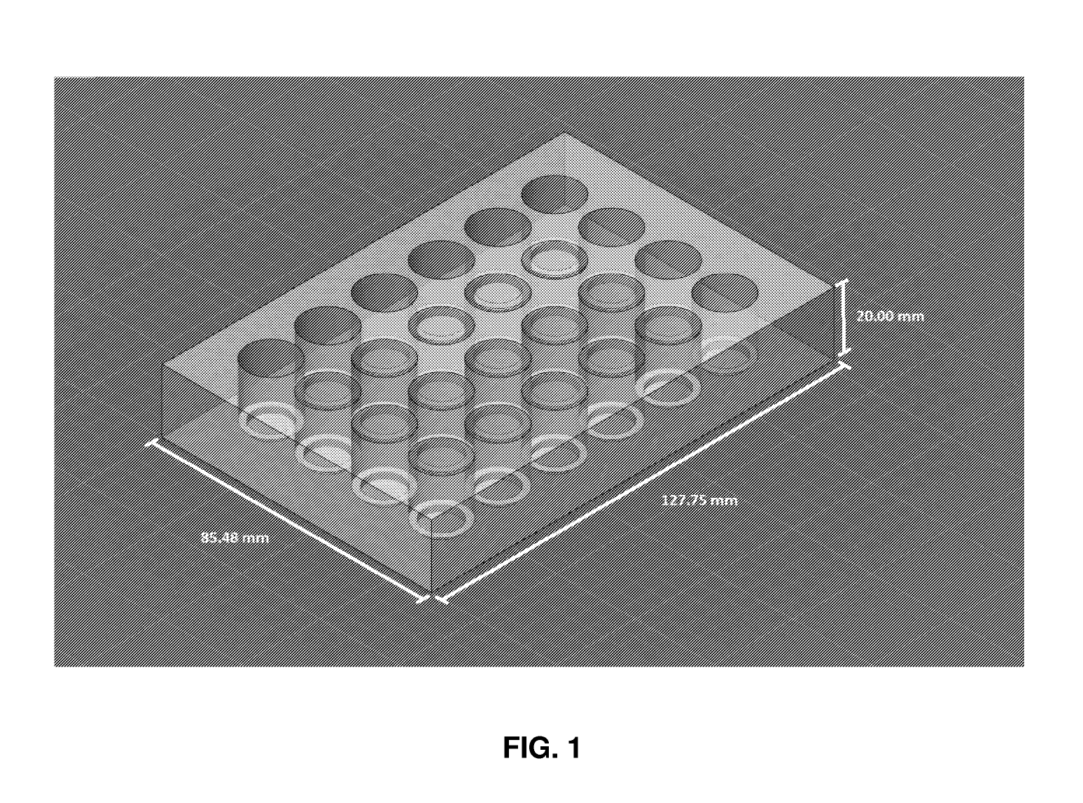

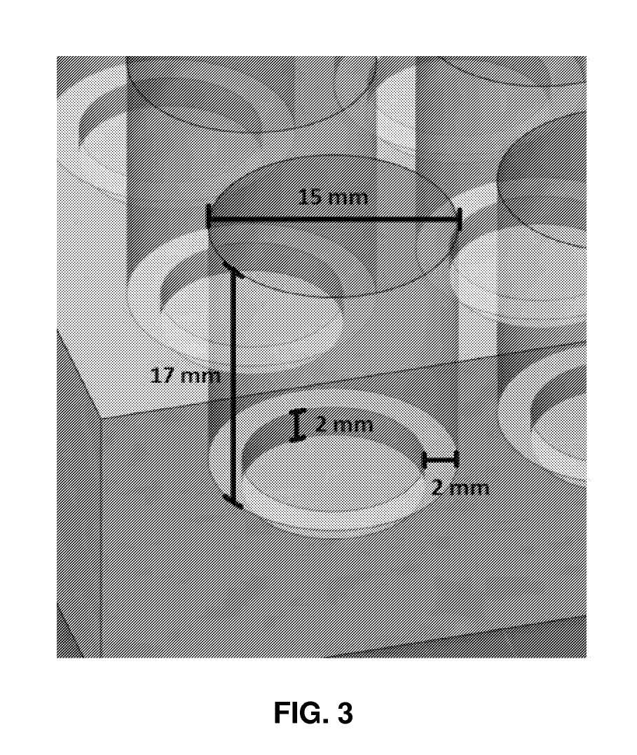

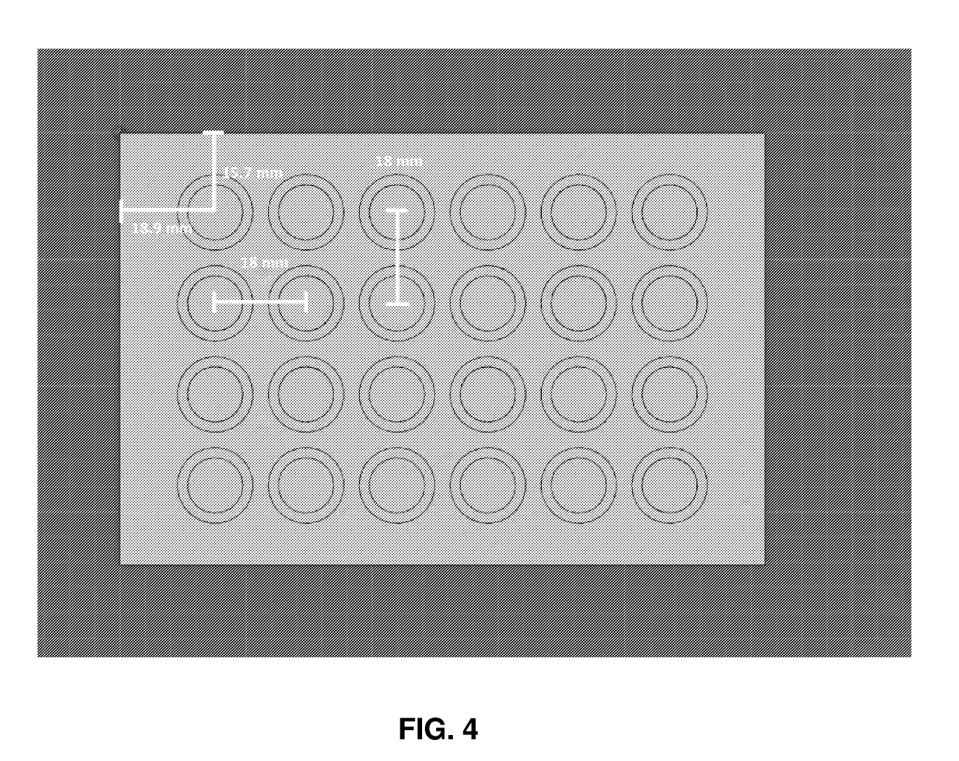

In accordance with one or more embodiments, a well plate may include a plurality of wells for use in any laboratory experiment, such as a cell culture experiment. The well plate may include any conventional number of wells, for example, 6, 12, 24, 48, 96, 192, 384, or 1536 wells. A wide variety of plates with differing numbers and sizes of wells are all within the scope of this disclosure. The well plates may be injection molded or manufactured by other conventional approaches. The well plates may be made of any conventional material but should generally be bio-inert, sterilizable, and relatively clear. In some non-limiting embodiments, the well plate may be made of polystyrene, polypropylene, polycarbonate, a cyclo-olefin, or a like material. In some embodiments, the well plate may be coated, plasma treated, or textured depending on its intended application. The wells may be modified or coated with various materials including, but not limited to, collagen, Matrigel™ matrix (Coming Incorporated), extracellular matrices, amino acids and growth factors. The well plate market is highly standardized in view of automation and microscopy considerations so it is generally desirable for the well plates to have conventional footprints, center-to-center distances, and offset distances as illustrated in the non-limiting embodiments of FIGS. 1-4, although the overall dimensions may have tolerances that allow for some variation.

In accordance with one or more embodiments, each well of the well plate may have an interior wall that includes a ledge positioned at a fixed depth within the well. The ledge may assist in a laboratory experiment, such as by facilitating media changes during a cell culture experiment as discussed below. The ledge may be constructed and arranged to deter substrate disruption during media changes. The ledge may be particularly important for 3D cell culture in which the cells are much easier to interfere with and disrupt when compared to 2D cell culture. By including the ledge, interference may be prevented by keeping the substrate and cells in a nonadjacent lower portion of the well, and allowing for pipetting to be performed easily and stress-free on the ledge.

In accordance with one or more embodiments, the ledge may generally be defined as an indentation within the interior wall or as an extension of the interior wall. In some embodiments, the ledge may protrude into the well along at least a portion of a circumference of the interior wall. In some embodiments, the ledge may protrude a uniform distance into the well along at least a portion of the circumference of the interior wall. The ledge surface may generally be horizontal or at some desired angle. In at least some embodiments, the ledge may protrude into the well along an entire circumference of the interior wall to define a first well zone above the ledge and a second well zone below the ledge. The second well zone may have a smaller diameter than the first well zone. In some embodiments, the smaller volume or surface area in the lower zone may be substantially negligible in terms of impact on experimental parameters but may be offset through adjustment of substrate volume or thickness. The second well zone may have a substantially flat bottom and may be configured to receive a substrate during the cell culture experiment. Other geometries such as a conical shape are within the scope of the disclosure.

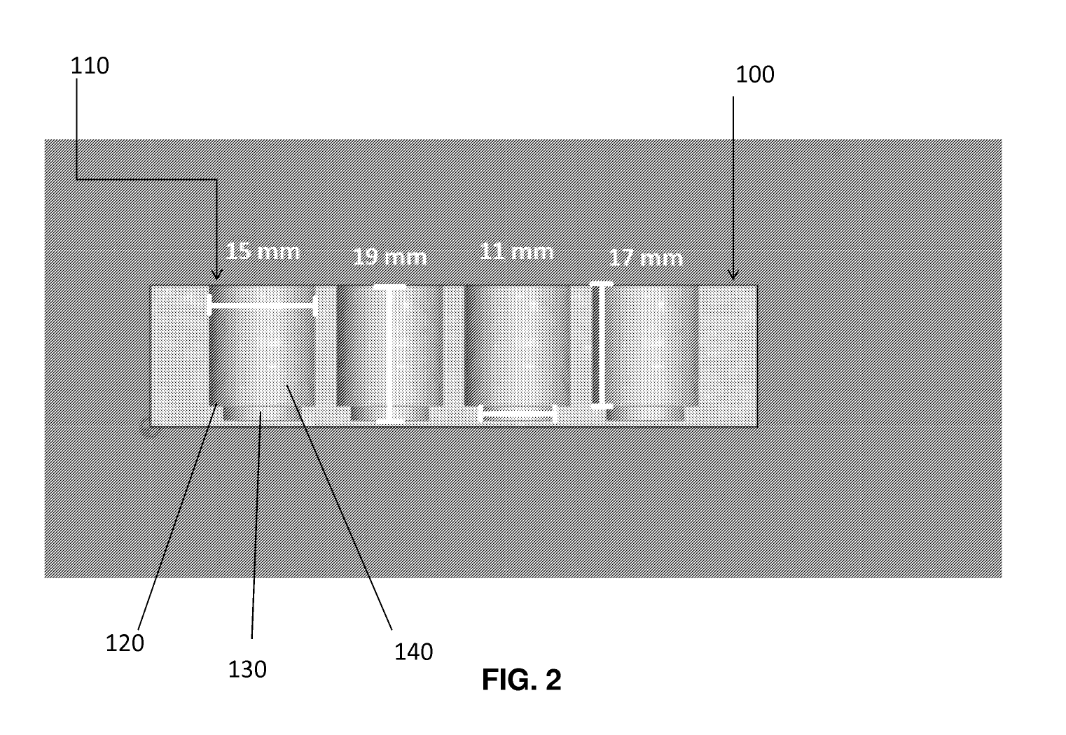

As illustrated in the non-limiting embodiment of FIG. 2, well plate 100 may include multiple wells 110. An interior wall of well 110 may include a ledge 120 to create a step within well 110. Ledge 120 may define an lower well zone 130 and an upper well zone 140.

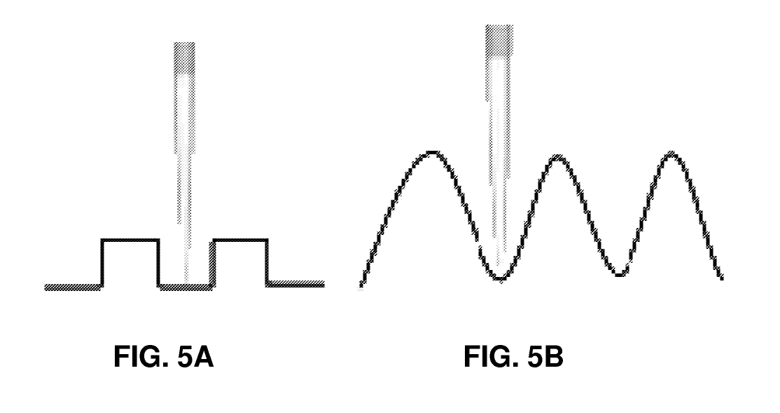

In accordance with one or more embodiments, the ledge may generally define a platform upon which a pipette tip may be applied. In some embodiments, the ledge may include a substantially flat, horizontal surface. In other embodiments, the ledge may be an angled surface. In still other embodiments, the ledge may include a contoured surface or shape. FIGS. 5A-5B present views of non-limiting ledge contours in accordance with some embodiments. Other ledge geometries constructed and arranged to provide a resting surface for a pipette tip are within the scope of the disclosure.

In accordance with one or more embodiments, the ledge may define a substantially cylindrical step in the well between a lower well zone and an upper well zone. In at least some embodiments, the ledge or step is configured to provide a platform for a pipette tip in a location that is nonadjacent to the substrate during the cell culture experiment. In some non-limiting embodiments, the ledge should generally be wide enough to accommodate a pipette tip and to provide a resting position for the pipette tip. The ledge should also be wide enough to distance a cell culture substrate from a pipette tip during a media change operation to prevent disruption thereof. Thus, the type of pipette tip and dimensions of the substrate are design factors. In some non-limiting embodiments, the ledge may protrude between about 0.5 mm and about 5 mm into the well along at least a portion of the circumference of the interior wall. In at least one non-limiting embodiment, the ledge may protrude about 2 mm into the well along at least a portion of the circumference of the interior wall. Other ledge widths are within the scope of the disclosure.

In accordance with one or more embodiments, the ledge may be positioned at a fixed depth within the well as noted above. The fixed depth may be related to a predetermined volume of media used during a cell culture experiment or other experimental design parameter. It may be generally desirable to keep a cell culture substrate submerged during a media change. The well plate can thus be designed such that the ledge is positioned a fixed distance above the cell culture substrate. In some non-limiting embodiments, substrates may generally be about 1.25 mm thick and thus the fixed depth of the ledge may therefore be about 2 mm from a bottom of the well to ensure submersion. During a media change, spent media can be aspirated down to the ledge to leave a predetermined volume of spent media within the well prior to replenishing. Overall dimensions for the depth and width of the ledge or step may be optimized and generally informed based on the nature of the experiment and substrate. Beneficially, each well of the well plate can have similar dimensions for consistency.

In some non-limiting embodiments, wells of a multiwell plate may contain a circumferential indentation of a specified distance in the direction of the center of the well, beginning at the bottom of the well and ending at a specified distance from the top of the well, thereby creating a cylindrical step at the bottom of the well. In some non-limiting embodiments, the step height may be between about 0.1 mm and 10 mm. In at least one non-limiting embodiment, the step height may be about 2 mm. Other step heights are within the scope of the disclosure. The step may provide a platform for the placement of a pipette or aspirator tip in a location that is nonadjacent from the cell culture substrate, increasing the ease, speed and accuracy of media changes while greatly reducing the potential for disruption of the cell culture substrate. In some embodiments, the wells of a well plate may include a continuous horizontal ledge. Any variations in terms of well number, any less than full circumference of ledge, any other ledge angle, and any other types of wells are all viable embodiments.

In accordance with one or more embodiments, a substrate, such as a collagen scaffold or a hydrogel may be placed in each well of the well plate during use. In at least some embodiments, the substrate may be positioned in a lower zone of a well as defined by the step or ledge discussed above. The substrate may be generally seeded with cells for 2D or 3D cell culture. Media may provide nutrients to the cells and may be replenished periodically in each well.

In accordance with one or more embodiments, a method of changing media in a well of a well plate during a cell culture experiment may involve introducing a pipette tip to the well, and tracing an interior wall of the well with the pipette tip from a top of the well to a ledge positioned at a fixed depth within the well. The pipette tip can then be rested on the ledge and a predetermined volume of spent media may be aspirated from the well with the pipette tip based on the fixed depth of the ledge. For example, spent media may be aspirated from the well until the level of remaining spent media in the well is flush with the ledge thus dictating a remaining volume of spent media. Beneficially, such volume may be substantially uniform across all wells of the well plate due to the structure of the wells including the ledges. A predetermined volume of fresh media may then be introduced to the well without disrupting the substrate which is located nonadjacent to the ledge within the well. The media change operation may therefore be simplified for a user in various embodiments, eliminating guess work, particularly if the ledge continues along an entire circumference of the well such that operation can be largely by feel rather than visualization. In some embodiments, the method may involve the use of a single pipette tip. In other embodiments, the method may involve the use of two or more pipette tips, such as one pipette tip for aspirating spent media and a second pipette tip for introducing fresh media.

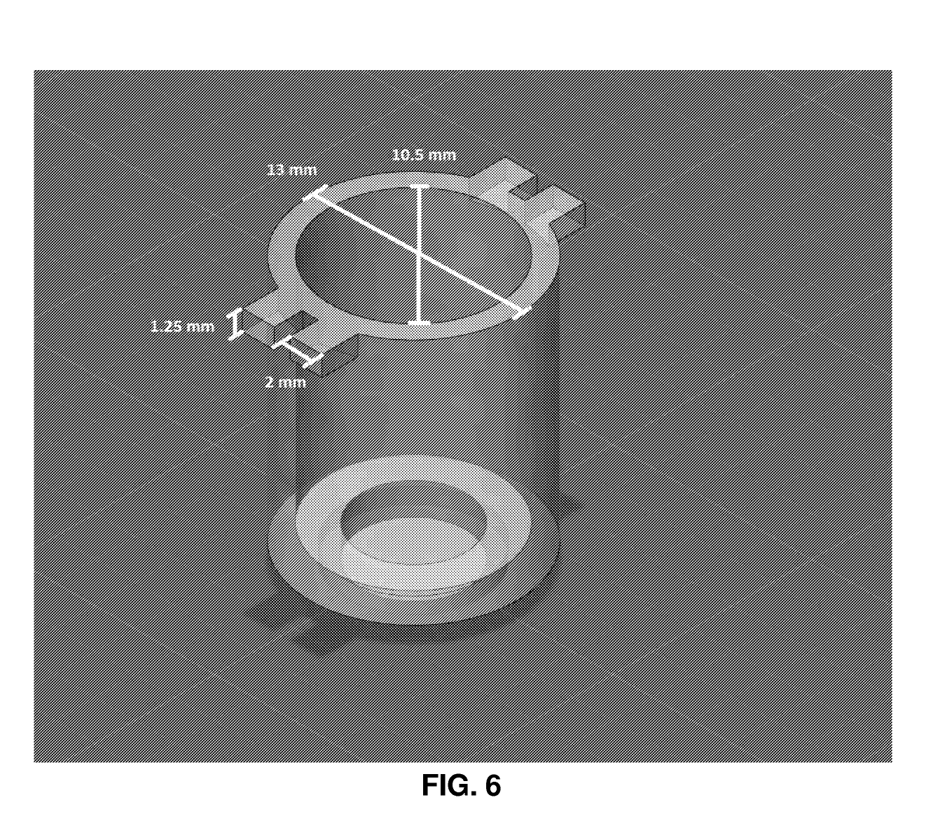

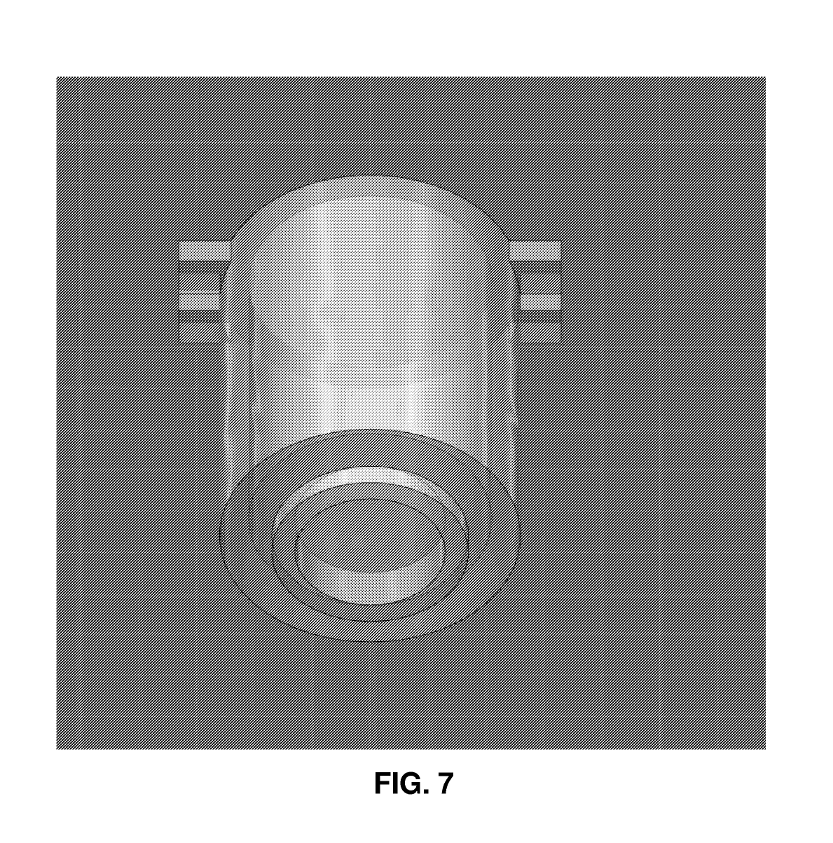

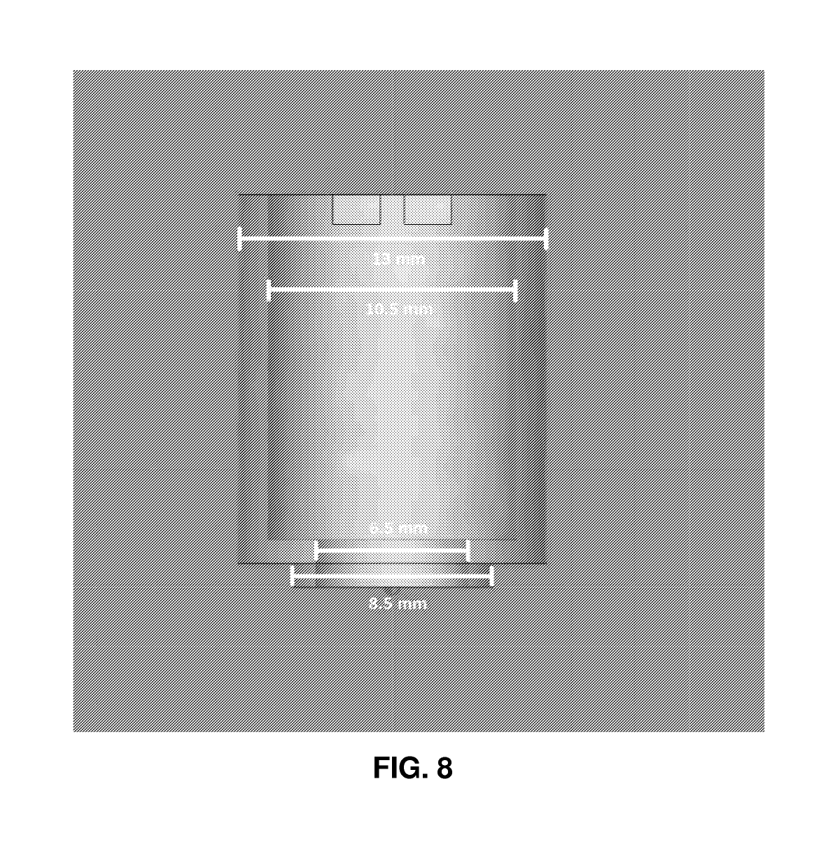

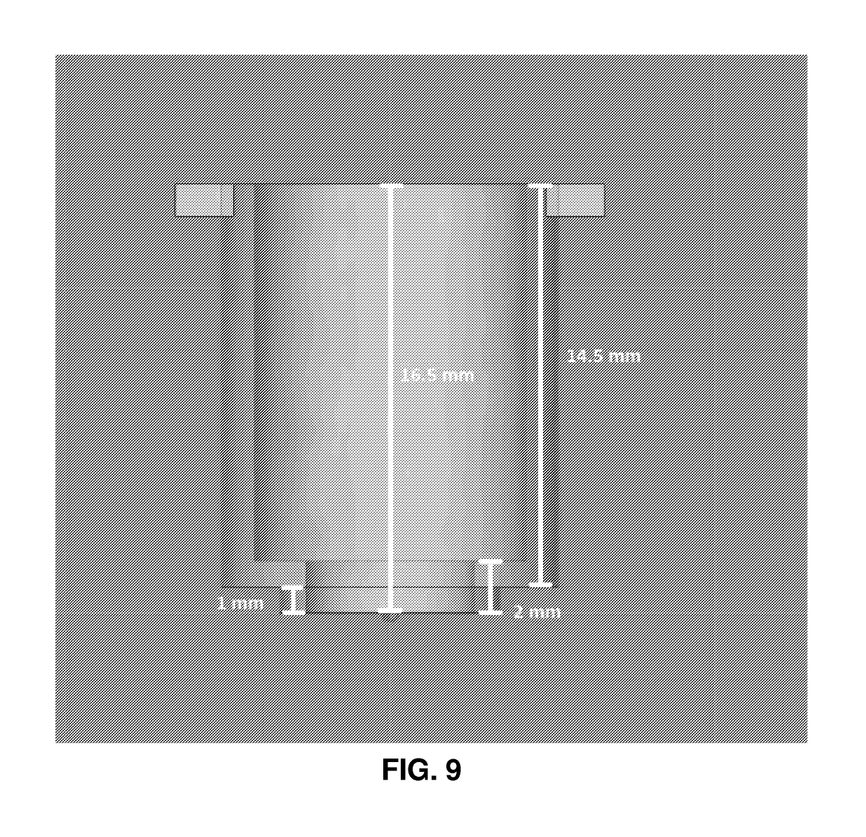

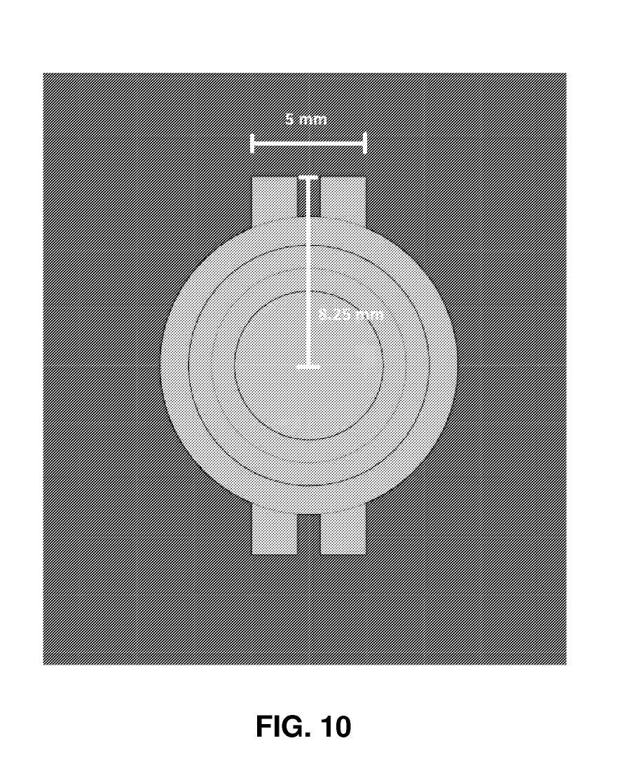

In accordance with one or more embodiments, a well plate accessory may include an insert for a well of a conventional well plate. Thus, a standard or conventional well plate may be retrofitted to facilitate laboratory experiments, such as cell culture experiments as described herein. The use of inserts may generally increase the interval between media changes in some embodiments. An insert may be constructed and arranged to provide a ledge at a fixed depth within the well to facilitate media changes during a cell culture experiment. In some embodiments, conventional inserts may be modified to include a step or ledge. Sizing and construction of the insert may generally be designed to provide a desired surface area to grow cells. In some non-limiting embodiments, an outer diameter of an insert may be adjusted to allow for the step or ledge. In some embodiments, the wall thickness may be kept substantially uniform. FIGS. 6-10 present various schematics of inserts in accordance with one or more embodiments, including non-limiting sample dimensions for illustrative purposes only to facilitate the integration with conventional well plates and associated equipment.

In some embodiments, an insert may include a permeable bottom. In at least some embodiments, a membrane may be generally positioned at the bottom of an insert to provide permeability. Other materials fall within the scope of the disclosure. The type of membrane material and its pore size may vary depending on intended use such as desired optical properties and the type of cells to be plated. Some conventional membrane materials include polyester (PET), polycarbonate (PC), and collagen-coated polytetrafluoroethylene (PTFE). Some common pore sizes are 0.4 μm, 1.0 μm, 3.0 μm, 5.0 μm and 8.0 μm. Choice among these different pore sizes generally depends on the type of experiment that is being performed, such as drug transport studies or cell migration. In at least some other embodiments, the permeable bottom may be made of a plastic material that can be rendered permeable via modification. The permeable bottom may be, for example, a mesh, or non-woven material in addition to conventional membrane materials.

In accordance with one or more embodiments, the inserts may hang on the well plate. In other embodiments, holders may keep the insert elevated above the bottom of a well. In some embodiments, inserts may lock onto the well plate to prevent the inserts from sliding or shifting around inside the well. Capillary action enabled by close contact between the well plate and insert wall may change the effective depth of media around the insert. It is generally desirable for the media to remain in contact with the bottom of the insert. Air bubbles on the bottom of the insert and detachment due to capillary action if the insert is too close to the side of the well may therefore be problematic because lack of contact between media and substrate could invalidate an experiment for reasons such as lack of nutrients or potential adverse reactions by the cell substrate when exposed to air. In some embodiments, the insert may be made from a material that prevents or reduces capillary action. In at least some embodiments, the material of the insert may be substantially hydrophobic and non-wettable. In some non-limiting embodiments, there may be an appreciable distance between the bottom of the insert and the bottom of the well.

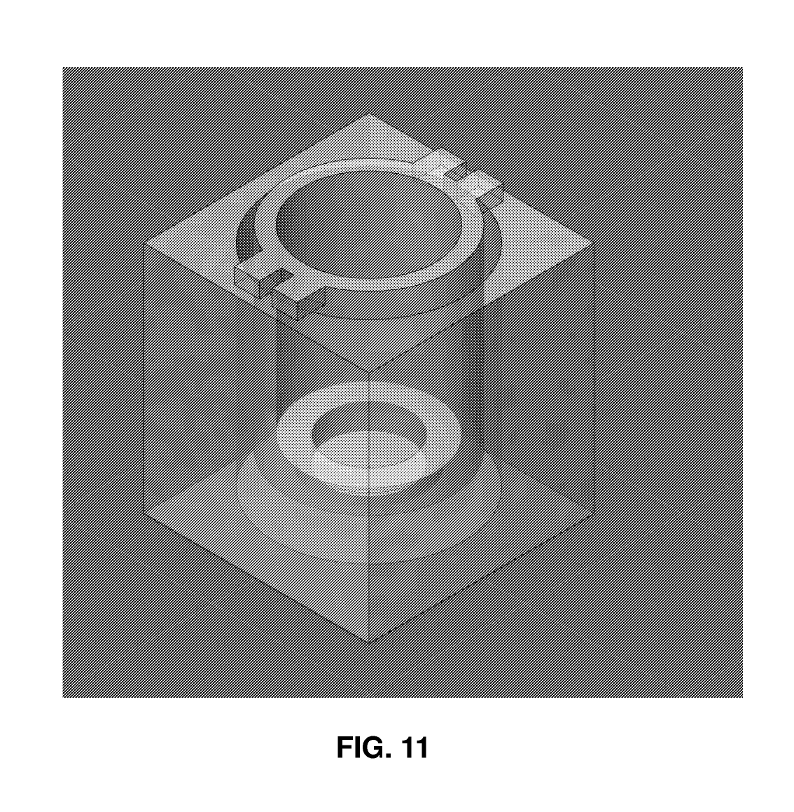

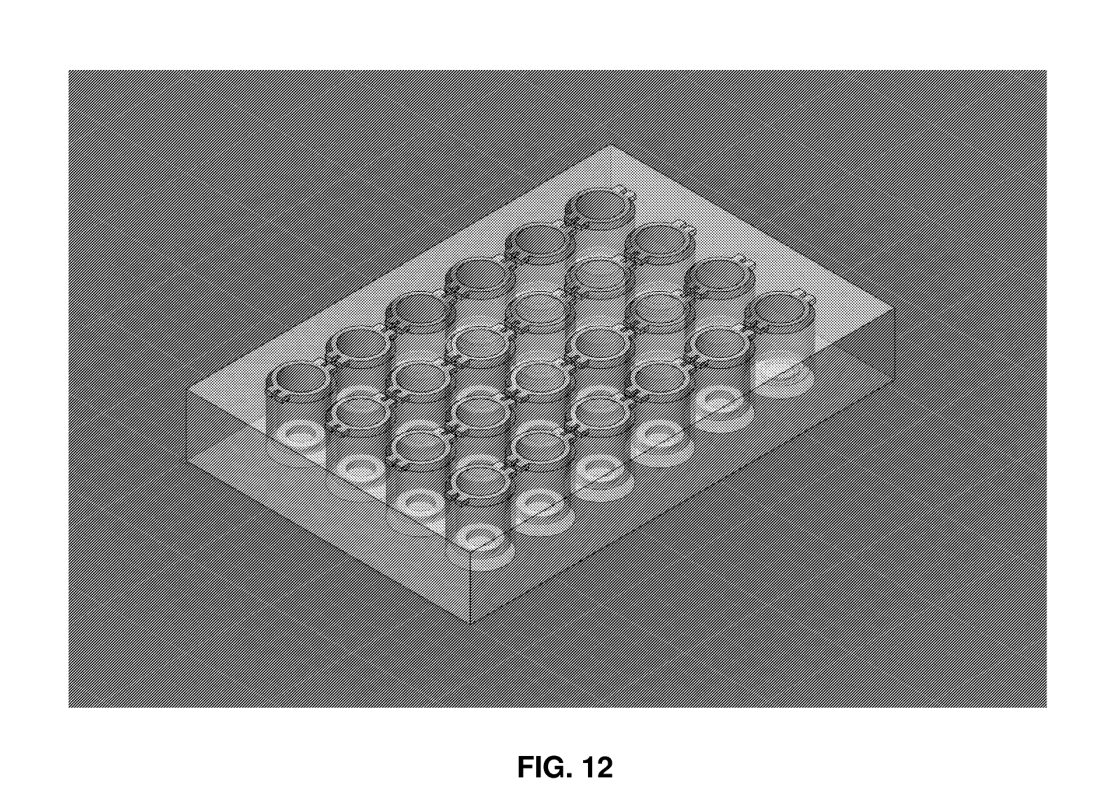

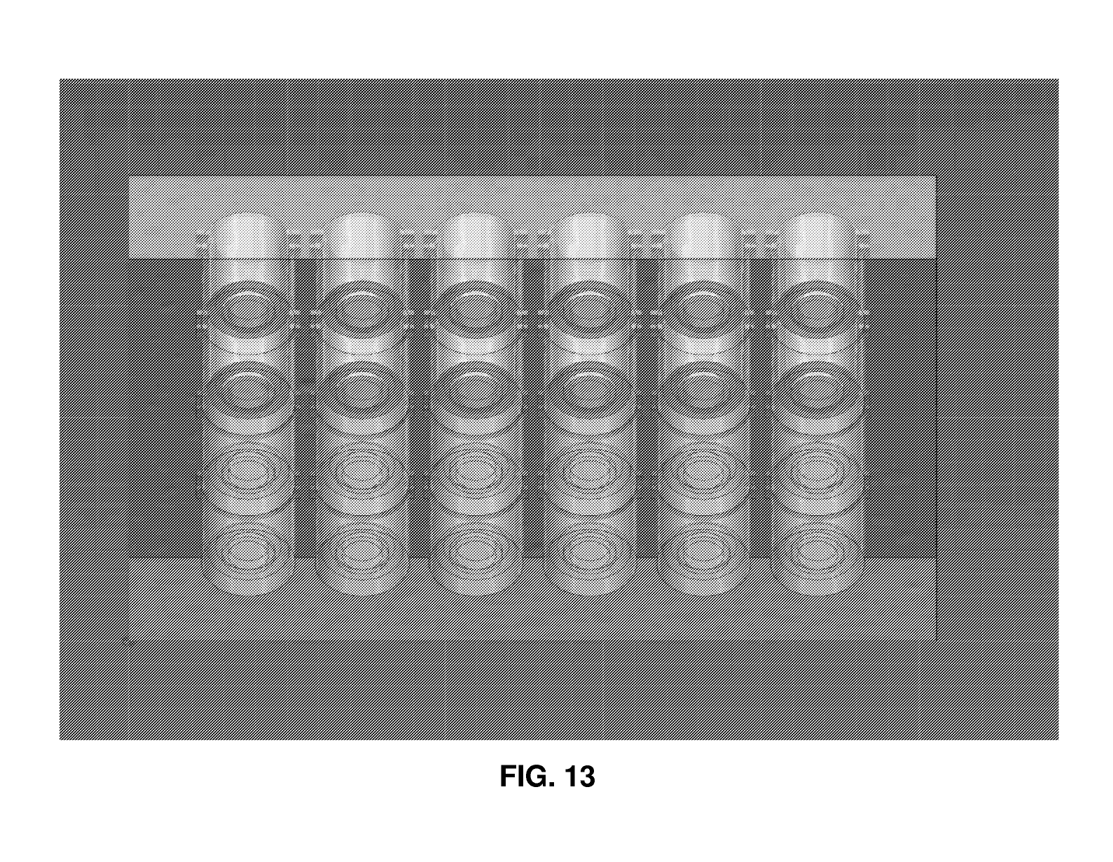

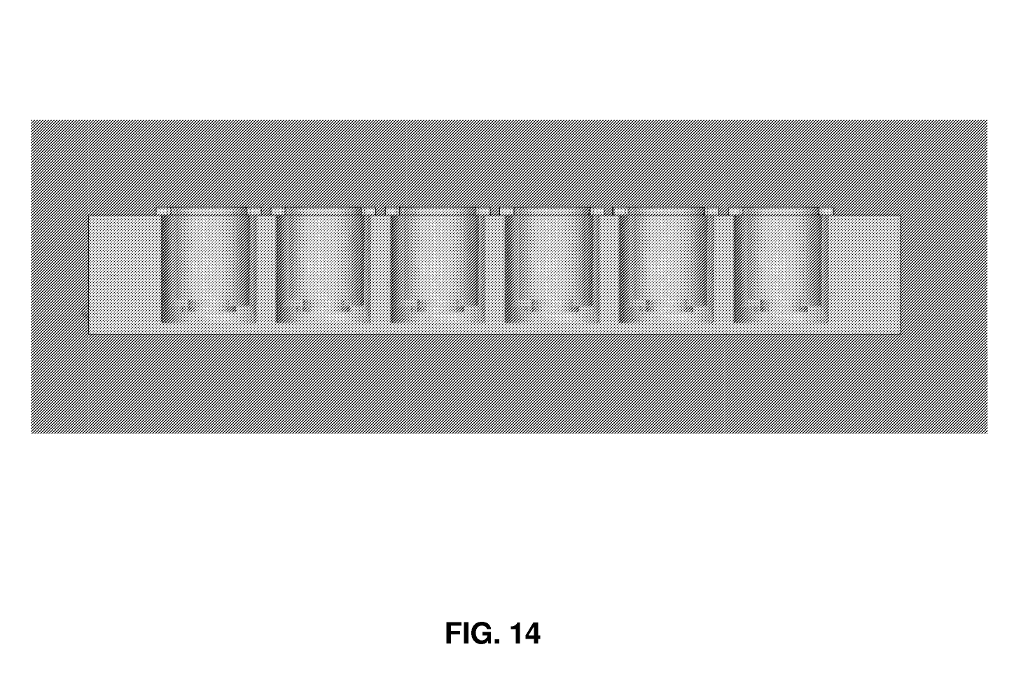

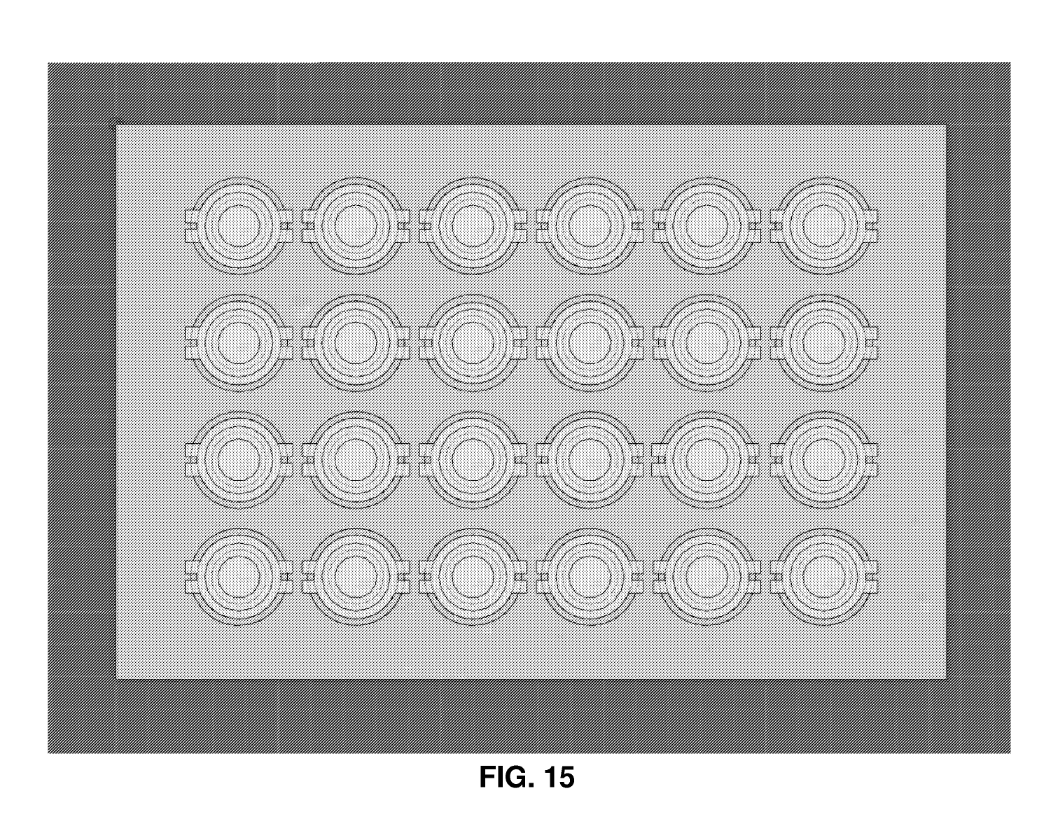

FIG. 11 presents a schematic view of the placement of a single insert within a segment of a well plate. FIGS. 12-15 generally present schematics of well plates in which wells are filled with inserts in accordance with one or more embodiments.

In accordance with one or more embodiments, a plurality of inserts may be connected in an array configured to be received across multiple wells of a well plate. In some embodiments, an array of inserts may be constructed and arranged to be easily broken apart. An array of inserts may be manipulated as a single unit to facilitate automation of labware.

In some embodiments, individual inserts may be marked with a unique identifier such that they may remain distinct, even if moved to a different position in an array of wells as an experiment progresses. This may allow for the mix and match of inserts, for example, the consolidation of inserts into a smaller number of well plates as an experiment progresses and certain inserts are deemed not qualified for continuation due to contamination or other human errors. A mechanism of identification may also be useful if the inserts are removed and handled individually for reasons such as microscopy. In some embodiments, various items of information may provide a unique code scheme and may be used in combination. These include date, manufacturing machine, number in a series of inserts created, or a quality control number.

In accordance with one or more embodiments, a cell culture kit may include a well plate having a plurality of wells, each well having an interior wall that includes a ledge positioned at a fixed depth within the well as described above. Other kits may include a conventional well plate and one or more inserts as described above to provide the ledge. The cell culture kit may further include a substrate, such as a hydrogel. A substrate, such as those described in U.S. Pat. No. 7,713,923 to Genove et al. which is hereby incorporated herein by reference in its entirety for all purposes, may generally provide a synthetic matrix to create a 3D environment for a cell culture experiment. In some embodiments, the hydrogel may be a PuraMatrix™ self-assembling peptide hydrogel commercially available from 3-D Matrix, Inc. and distributed by Corning Incorporated Life Sciences (MA). A kit may include any volume and concentration of substrate, such as a hydrogel. In some specific non-limiting embodiments, the kit may include a 5 ml vile of 1% PuraMatrix™ peptide hydrogel. In at least some embodiments, the hydrogel may include a repeating amino acid sequence of Arginine-Alanine-Aspartic Acid-Alanine. Other sequences of amino acids are within the scope of the disclosure, such as RADA16, IEIK13, KLD12, and other peptide hydrogels. In at least some embodiments, a substrate may include functionalized peptides. Other scaffold and cell culture substrate materials commonly known to those in the art such as conventional collagen or polymer-based hydrogels may also or alternatively be included. A substrate material may be provided at an adjusted pH level, isotonicity, or other relevant parameter. Kits may otherwise include instructions and materials for such alteration.

In accordance with one or more embodiments, a cell culture kit may further include cell specific protocols such as those regarding surface planting and/or encapsulation for 2D and/or 3D cell culture experimentation. A kit may include instructions for conducting a media change in a well of the well plate while deterring disruption of the substrate during a cell culture experiment. The methods may prevent cell disruption, such as monolayer disruption and/or membrane damage while performing manual assays. In some embodiments, the kit may further include a source of media. In some embodiments, a buffering solution may be provided. In other embodiments, the kit may include labware such as pipettes and/or other instrumentation. In at least some embodiments, a kit may include one or more reagents such as lyophilized sucrose. A cell culture kit may also include media and media dye. Some kits may include extracellular matrix (ECM) supplements such as growth hormones and proteins.

In accordance with one or more embodiments, a method of facilitating cell culture may involve providing a well plate including a plurality of wells, each well having an interior wall that includes a ledge positioned at a fixed depth within the well as discussed above. The method may further involve providing instructions for conducting a media change in a well of the well plate during a cell culture experiment. In some embodiments, the instructions may involve the use of a single pipette tip. In other embodiments, the instructions may involve the use of two or more pipette tips, such as a first pipette tip for aspirating spent media and a second pipette tip for introducing fresh media. In some embodiments, the method may still further involve providing a substrate for use during the cell culture experiment.

In accordance with one or more embodiments, a well plate and/or accessory may include indicia, such as color coding or symbols, to facilitate a laboratory experiment. In accordance with one or more embodiments, the well plates may be constructed and arranged to be generally compatible with existing equipment and to interface with complementary technologies, such as cell culture automation and robotics, microscopes and automatic plate readers.

It is to be appreciated that the embodiments discussed herein are not limited in application to the details of construction and the arrangement set forth herein. The aspects are capable of implementation in other embodiments and of being practiced or of being carried out in various ways. Examples of specific implementations are provided herein for illustrative purposes only and are not intended to be limiting. In particular, acts, elements and features discussed in connection with any one or more embodiments are not intended to be excluded from a similar role in any other embodiment.

Also, the phraseology and terminology used herein is for the purpose of description and should not be regarded as limiting. The use herein of “including,” “comprising,” “having,” “containing,” “involving,” and variations thereof is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be part of this disclosure and are intended to be within the scope of the invention. Accordingly, the foregoing description and drawings are by way of example only.

What is claimed is:

1. A well plate, comprising:
a plurality of wells, each well having an interior wall that includes a ledge positioned at a fixed depth within the well to facilitate media changes during a cell culture experiment.

2. The well plate of claim 1, wherein the fixed depth is related to a predetermined volume of media used during the cell culture experiment.

3. The well plate of claim 2, wherein the fixed depth is in a range of about 0.1 mm to about 10 mm from a bottom of the well.

4. The well plate of claim 1, wherein the ledge is constructed and arranged to deter substrate disruption during media changes.

5. The well plate of claim 1, wherein the ledge protrudes into the well along at least a portion of a circumference of the interior wall.

6. The well plate of claim 5, wherein the ledge protrudes a uniform distance into the well along at least a portion of the circumference of the interior wall.

7. The well plate of claim 6, wherein the ledge protrudes from about 0.5 mm to about 5 mm into the well along at least a portion of the circumference of the interior wall.

8. The well plate of claim 5, wherein the ledge protrudes into the well along an entire circumference of the interior wall to define a first well zone above the ledge and a second well zone below the ledge.

9. The well plate of claim 8, wherein the second well zone has a smaller diameter than the first well zone.

10. The well plate of claim 9, wherein the second well zone has a substantially flat bottom and is configured to receive a substrate during the cell culture experiment.

11. The well plate of claim 9, wherein the ledge defines a substantially cylindrical step in the well between the first and second well zones.

12. The well plate of claim 10, wherein the ledge is configured to provide a platform for a pipette tip in a location that is nonadjacent to the substrate during the cell culture experiment.

13. The well plate of claim 1, wherein the well plate includes 6, 12, 24, 48, 96, 192, 384, or 1536 wells.

14. A cell culture kit, comprising:
a well plate according to claim 1;
a substrate; and
instructions for conducting a media change in a well of the well plate while deterring disruption of the substrate during a cell culture experiment.

15. The cell culture kit of claim 14, further comprising a source of media.

16. The cell culture kit of claim 14, wherein the substrate comprises a hydrogel that includes a repeating amino acid sequence of Arginine-Alanine-Aspartic Acid-Alanine.

17. A method of changing media in a well of a well plate during a cell culture experiment, comprising:
introducing a pipette tip to the well;
tracing an interior wall of the well with the pipette tip from a top of the well to a ledge positioned at a fixed depth within the well;
resting the pipette tip on the ledge;
aspirating a predetermined volume of spent media from the well with the pipette tip based on the fixed depth of the ledge; and
introducing a predetermined volume of fresh media to the well without disrupting a substrate located nonadjacent to the ledge within the well.

18. A method of facilitating cell culture, comprising:
providing a well plate according to claim 1; and
providing instructions for conducting a media change in a well of the well plate during a cell culture experiment.

19. The method of claim 18, further comprising providing a substrate for use during the cell culture experiment.

20. A well plate accessory, comprising:
an insert for a well of a well plate, the insert constructed and arranged to provide a ledge at a fixed depth within the well to facilitate media changes during a cell culture experiment.
